(19) United States
(12) Patent Application Publication
(10) Pub. No.: US 2025/0387063 A1
(43) Pub. Date: Dec. 25, 2025

(54) OPTIMIZED ELECTROCARDIOGRAM (ECG) AND ACTIVITY DATA VISUALIZATION

(71) Applicant: KONINKLIJKE PHILIPS N.V., Eindhoven (NL)

(72) Inventors: Wilhelmus Johannes Joseph Stut, Eindhoven (NL); Marie Perez, Amsterdam (NL); Pien Schaeffers, Eindhoven (NL)

(21) Appl. No.: 19/245,635

(22) Filed: Jun. 23, 2025

Related U.S. Application Data
(60) Provisional application No. 63/663,771, filed on Jun. 25, 2024.

(51) Int. Cl. A61B 5/339; A61B 5/00

(52) U.S. Cl. CPC A61B 5/339; A61B 5/0006; A61B 5/725

(57) ABSTRACT

An embodiment includes a system having an ambulatory patient sensor, such as a cardiac sensor, that provides data via a network to remote device configured to prepare and display interactive user interface(s) used for visualizing data, for example in an interactive web application. The interactive user interface(s) are provided with interactive element(s) configured to support data sort or filtering operations for visualizing patient metrics and data activity included in the data visualization. In an embodiment, the interactive user interfaces provide different filtered views of the data responsive to user interaction for easily transitioning between data visualizations in an intuitive manner.

### CROSS-REFERENCE TO RELATED APPLICATIONS

This patent application claims the priority benefit under 35 U.S.C. § 119(e) of U.S. Provisional Application No. 63/663,771, filed on Jun. 25, 2024, the contents of which are herein incorporated by reference.

### BACKGROUND

### 1. Field

The disclosed subject matter generally pertains to data visualization. Certain disclosed subject matter relates to interactive user interfaces for visual data mining or browsing structured data.

### 2. Description of the Related Art

Mobile cardiac telemetry systems gather electrocardiogram (ECG) data in ambulatory settings (e.g., in the home environment or on-the-go). An example of mobile cardiac telemetry is use of an ambulatory patient sensor placed in a patch on the chest. The ambulatory patient sensor communicates wirelessly with a personal device, for example a smartphone having a dedicated mobile application (“app”), which is in turn connected (via the mobile phone network) to a remote system, such as a clinical service center. An example of such a mobile cardiac telemetry system is the Philips BioTel mobile cardiac outpatient telemetry (MCOT) system.

As it is useful to relate arrhythmic events to symptoms, patients are encouraged to inform the system when they feel symptoms. Patients are also asked to describe their symptoms as well as the activity they were engaged in when symptoms presented, which is typically done via an app on a smartphone. For example, when patients feel a symptom, they select the symptoms recording function of the app, and provide details like the nature of the symptom (e.g., fainted, dizzy, chest pain, etc.) and the activity (and/or intensity of the activity) they were doing when the symptom occurred. The current date and time are recorded automatically.

### SUMMARY

Sorting data for display within data visualization applications assists users in understanding and appreciating clinically important points within the data. For example, various sort operations may be applied to a data visualization to determine whether independently collected data is (or is not) notable with respect to an event of interest, such as an event of interest to a clinician (“event” or “clinical event”). Current approaches to visualizing cardiac medical data, however, often do not match user expectations. For example, current approaches to sorting cardiac medical data fail to present data marks in a coordinated, clinically relevant fashion and at differing levels of granularity. Because of this, users are sometimes required to take unintuitive steps to produce a desired data visualization or may be unaware that the data, if sorted and visualized differently, is clinically relevant.

An embodiment provides user interfaces with a capability to perform sort or filter operations that sort data marks within different sections independently of other sections within the same data visualization, while coordinating the display of the variously sorted data marks among related sections to provide added context, for example relating different data visually using time of collection. An embodiment therefore facilitates a user sorting data marks at differing levels of granularity within the same interactive display interface. As will be explained in greater detail below, sort operations apply data filter(s) to the data, allowing for data marks within different sections of a data visualization to be independently ordered and displayed in a coordinated fashion.

In summary, an embodiment provides system, comprising an ambulatory patient sensor configured to be coupled to a patient and sense data for a continuous patient metric and activity data of the patient, and a local device in communication with the ambulatory patient sensor. A remote device is provided in communication with the local device and a display. In an embodiment, a database is implemented in a non-transitory media, and the remote device includes a set of one or more processors in communication with the database. In an embodiment, the set of one or more processors is configured to store the data for a continuous patient metric and the activity data of the patient in the database, identify an event based on one or more of the data for a continuous patient metric and the activity data of the patient, and display, in a user interface provided at the display, an initial contextual display associated with the event and comprising an initial type of continuous patient metric and the activity data of the patient. In an embodiment, the set of one or more processors is configured to apply, based on interaction with the user interface, one or more data filters to the data for a continuous patient metric, and thereafter provide an updated contextual display in the user interface. In an embodiment, the updated contextual display comprises a first section having a subset of the data for a continuous patient metric aligned in time with a subset of the activity data in a second section.

In an embodiment, the first section of the updated contextual display comprises an ECG strip and an ECG fragment encompassing the ECG strip. In an embodiment, the ECG strip and ECG fragment are aligned in time with the subset of the activity data associated with the event.

In an embodiment, the set of one or more processors is configured to: respond to user interaction with the updated contextual display by visually indicating a second subset of the activity data; and applying the one or more data filters to update the first section to display a second subset of the data for a continuous patient metric associated with the second subset of the activity data.

In an embodiment, the second section comprises a plurality of data marks segmented according to time and displayed in areas of predetermined activity intensity. In an embodiment, the set of one or more processors is configured to respond to user interaction with one of the plurality of data marks by applying the one or more data filters to select and display updated data for the first section.

In an embodiment, the updated contextual display comprises a second type of continuous patient metric, different than the initial type, displayed responsive to the interaction with an indicator of the event. In an embodiment, the initial type of continuous patient metric comprises one or more of heart rate data and blood pressure data, and the second type of continuous patient metric comprises electrocardiogram (ECG) data. In an embodiment, the initial contextual display comprises an initial time filter applied to the activity data; and the updated contextual display comprises the activity data filtered using a second time filter, different than the initial time filter, displayed responsive to the interaction with the indicator of the event.

In an embodiment, the ambulatory patient sensor comprises a motion sensor; and the activity data comprises data derived from the motion sensor.

In an embodiment, the set of one or more processors is configured to display, responsive to user interaction with the initial contextual display, ECG data associated with the event as an overlay on the initial contextual display. In an embodiment, the set of one or more processors is configured to display, responsive to user interaction with a part of the data for a continuous patient metric, electrocardiogram (ECG) data associated with the part.

In an embodiment, the event comprises one or more of a type of cardiac event and a discordance between the data for a continuous patient metric and the activity data. In an embodiment, the set of one or more processors is configured to display two or more indicators of events in the initial contextual display.

An embodiment provides a method, comprising: obtaining, from an ambulatory patient sensor configured to be coupled to the patient, data for a continuous patient metric and activity data of the patient; identifying, using a set of one or more processors, an event based on one or more of the data for a continuous patient metric and the activity data of the patient; displaying, in a user interface, an initial contextual display associated with the event and comprising an initial type of continuous patient metric and the activity data of the patient; applying, using the set of one or more processors, based on interaction with the user interface, one or more data filters to the data for a continuous patient metric; and thereafter providing, using the set of one or more processors, an updated contextual display in the user interface comprising a first section having a subset of the data for a continuous patient metric aligned in time with a subset of the activity data in a second section.

An embodiment provides a computer program product comprising code executable by a set of one or more processors to perform one or more of the methods, or part thereof, as described herein.

The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of a claimed invention.

### BRIEF DESCRIPTION OF THE DRAWINGS

FIG. 1 illustrates an example system according to an embodiment.

FIG. 1A illustrates example method according to an embodiment.

FIG. 2 illustrates an example user interface according to an embodiment.

FIG. 3 illustrates an example user interface according to an embodiment.

FIG. 4 illustrates a diagram of example system components according to an embodiment.

### DETAILED DESCRIPTION OF EXEMPLARY EMBODIMENTS

The described features, structures, or characteristics of the example embodiments may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

Current ECG data reports focus on arrhythmias but do not highlight moments without arrhythmia where a continuous patient metric, such as heart rate, and activity intensity are discordant (e.g., high heart rate while activity intensity is low) or more generally events where the data is otherwise of interest to a clinician, for example, when a patient reports sometimes having palpitations during high intensity activities, where a clinician may then want to visualize several high intensity activity episodes that may or may not have arrhythmias associated therewith. As a result, clinicians may miss situations that require clinical action, even absent arrhythmia detection. Also, current ECG reports do not relate ECG data to activity intensity in a graphically intuitive manner, which makes it difficult and time-consuming for clinicians to correlate these data.

Accordingly, an embodiment includes one or more interactive user interfaces comprising ECG report data. An embodiment provides a “macro” view (e.g., a 24-hour view) in which distinct pre-defined symbols (e.g., color coded exclamation marks) indicate different events, for example arrhythmias, discordant data, data associated with patient indications or reporting, or data otherwise of interest to a clinician for visualization (“events”).

An embodiment provides a “meso” view (e.g., a smaller time frame than the macro view, such as a 15-minute view) of the activity intensity around an event such as an arrhythmia. The location of the event in the meso view is indicated by way of a pre-defined indicator (e.g. a visual display such as a highlighted bar in an intensity graph of the activity data).

The description now turns to the figures. The illustrated example embodiments will be best understood by reference to the figures. The following description is intended only by way of example, and simply illustrates certain example embodiments.

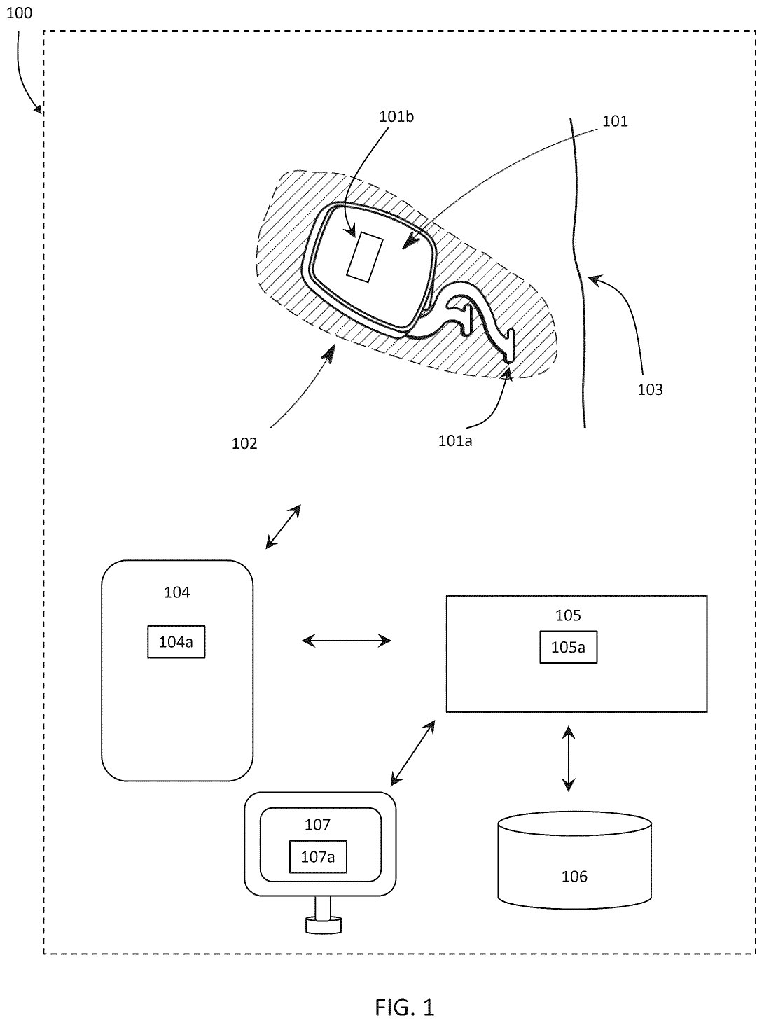

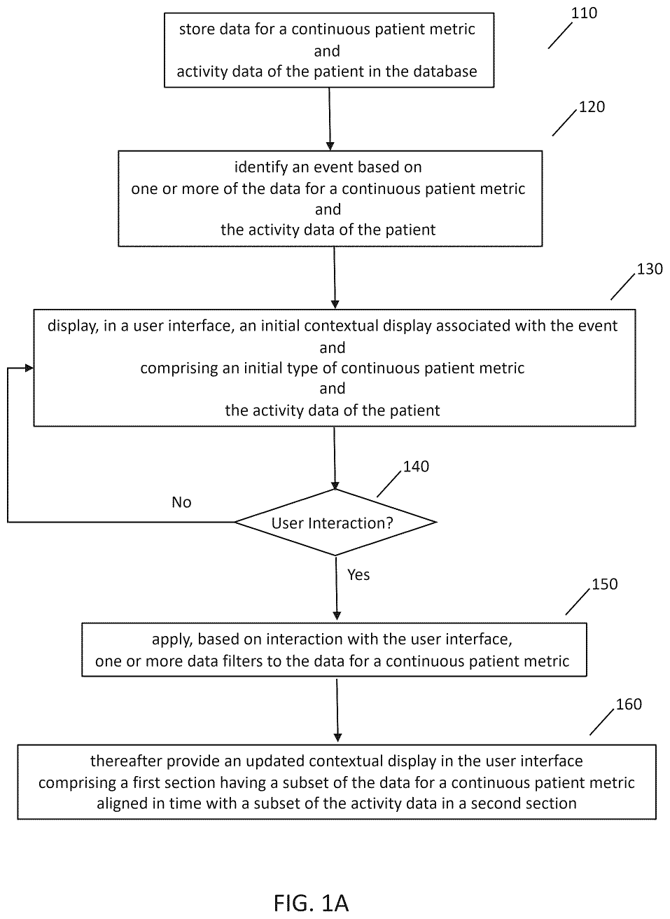

Illustrated in FIG. 1 is an example system 100 for mobile cardiac telemetry. System 100 includes an ambulatory patient sensor 101 that is adhered using a patch 102, e.g., adhesive, to a patient 103. Ambulatory patient sensor 101 in turn may include a variety of sensing devices, by way of example and not limitation, ECG sensor leads or electrodes 101a and a motion or an activity sensor 101b such as an accelerometer for detecting movement of ambulatory patient sensor 101 (and thus patient 103). Ambulatory patient sensor 101 may monitor, sense and report other continuous patient metrics, for example heart rate sensed by a heart rate sensor, blood pressure sensed by a blood pressure sensor, or similar. As such, ambulatory patient sensor 101 is configured to detect a continuous patient metric such as ECG data, heart rate, blood pressure or the like, as well as activity data, such as patient movement and the like, associated in time with the data of the continuous patient metric (“contextual” activity data). In an embodiment, more than one ambulatory patient sensor 101 may be utilized, or certain functionality of ambulatory patient sensor 101 may be provided by a distributed system, e.g., separate sensor devices cooperating to sense and report a continuous patient metric and activity data.

System 100 also comprises a local device 104 such as a personal user device, e.g., a smartphone or the like, with a module 104a configured to communicate, e.g., wirelessly, with ambulatory patient sensor 101. Local device 104 may also communicate, e.g., wirelessly, with a remote device 105, e.g., a server of a clinical system, which in turn operates a module 105a configured to communicate with a database 106 and display 107, such as a clinician display, which operates module 107a configured to communicate to one or more of local device 104 and remote device 105. In an embodiment, modules 104a, 105a, and 107a provided in local device 104, remote device 105, and display 107, respectively, may comprise computer code, software or firmware, such as applications, as well as a set of one or more processors, configured execute the code to process medical data such as a continuous patient metric and activity data, e.g., for storage in database 106 and visualization on an appropriate device, for example display device 107 or a suitable display device. It will be understood that system components may be combined as well as be distributed in system 100 variously.

In an embodiment, database 106 may store one or more of data for a continuous patient metric and activity data as structured data in combination with metadata. For example, database 106 may include data storage in the form of relational tables that store data derived from ambulatory patient sensor 101 in an organized manner, such as arranged in tables with metadata describing the stored data in columns and rows, e.g., by sensor type, activity type, metric type, time, etc. Database 106 may be queried, for example in response to user interaction with one or more interactive elements of a user interface, which may be configured to prepare and send one or more predetermined queries for data to database 106. Further, database 106 may respond to one or more queries with data for a continuous patient metric, activity data, and metadata. The metadata provided by database 106 may be used to sort or filter the data locally, e.g., apply a predetermined filter or sort operation to the data responsive to a user interaction with an interactive element.

Thus, ambulatory patient sensor 101 of system 100 is configured to be coupled to patient 103 and sense data for a continuous patient metric, e.g., heart rate and ECG data, and activity data, e.g., motion, of patient 103 for use in data visualization. System 100 includes remote device 105 in communication with ambulatory patient sensor 101, for example indirectly via local device 104, and remote device 105 includes or has access to database 106 implemented in a non-transitory media. Remote device includes module 105a, including a set of one or more processors in communication with database 106 to perform data visualization operations, such as described in FIG. 1A.

Referring to FIG. 1A, a set of one or more processors, e.g., of module 105a, is configured to store data for a continuous patient metric and activity data of patient 103 in database 106, as indicated at 110. The set of one or more processors of module 105a are configured to identify an event based on one or more of the data for a continuous patient metric and the activity data of the patient, as indicated at 120. For example, a clinical event such as an arrhythmia may be detected using signal processing applied to ECG data to identify ECG signal data characteristic of irregular heart rhythms. As another example, an event may be identified based on a discordance between activity data, such as high heart rate combined with, e.g., at the same time as, low intensity patient activity or motion. It will be understood that threshold(s) may be used to determine data discordance. In another example, an event may be identified through analysis of one or more types of data, as in the case of episodes of high intensity activity data identified via association with a patient report of intermittent palpitations during intense activity. In some examples, the identifying at 120 may be automated, for example based on arrhythmia detection, semi-automated, for example responsive to patient reporting data, or manual, for example in response to a user input such as a query for certain data like high intensity activity data.

In an embodiment, the set of one or more processors, e.g., of module 105a, are configured to provide data for display, in a user interface, of an initial contextual display associated with the event and comprising an initial type of continuous patient metric and the activity data of the patient, as indicated at 130. For example, the initial contextual display may be a “macro” display, as described herein, or an initial or first configuration of a “meso” display, as described herein.

In an embodiment, the set of one or more processors, e.g., of module 105a, is configured to apply, based on detecting an interaction with the user interface, as indicated at 140, one or more data filters to the data for a continuous patient metric, as indicated at 150. For example, a user interaction with a “macro” display having heart rate data aligned in time with contextual motion or activity data, as described herein, may include interaction with an indicator of an event, such as an arrhythmia or an indicator of a data discordance between activity data and data of a continuous patient metric. In an embodiment, the set of one or more processors, e.g., of module 105a, may thereafter provide an updated contextual display in the user interface comprising a first section having a subset of the data for a continuous patient metric aligned in time with a subset of the activity data in a second section, as indicated at 160. For example, in response to a user interaction with an event indicator displayed in a “macro” display, e.g., indicating an arrhythmia, the updated contextual display may filter the data for a continuous patient metric, e.g., heart rate data, to select and display a second type of continuous patient metric, e.g., ECG data, aligned in time with the activity data associated with the event.

In another example, the set of one or more processors, e.g., of module 105a, is configured to apply, based on interaction with the user interface, one or more different data filters to the data for a continuous patient metric, as indicated at 150. For example, a user interaction with a “meso” display having ECG data aligned in time with contextual motion or activity data, as described herein, may include interaction with a data mark for the contextual activity, such as a data mark for a time segment of activity data. In an embodiment, the set of one or more processors, e.g., of module 105a, may thereafter provide an updated contextual display in the user interface comprising a first section having a subset of the data for a continuous patient metric aligned in time with a subset of the selected activity data in a second section, as indicated at 160. For example, in response to a user interaction with a data mark in the contextual section of the “meso” display, e.g., indicating an interest in a preceding time segment of activity data, the updated contextual display may filter the data for a continuous patient metric, e.g., initial ECG data associated with another time segment of activity data, to select and display updated ECG data, aligned in time with the activity data selected.

From review of FIG. 1A, it will be understood that interactive user interfaces are provided with tools in the form of interactive elements, such as bars, zoom controls, and the like, displayed in the user interfaces that automatically or semiautomatically apply predetermined filter(s) to data, for example to display an initial data set organized around an event, and respond to user interaction by applying one or more other predetermined filters to update the display, based on the interactive element utilized. Filtering data may include querying different data, e.g., retrieved from database 106, for providing an updated display, locally filtering or sorting data already obtained from database 106, or a combination of the foregoing. Accordingly, an embodiment provides intuitive user interaction elements in user interfaces that filter and sort data for contextual display and visualization, permitting the user to easily navigate through a large amount of data without composing complex queries or applying numerous filters manually.

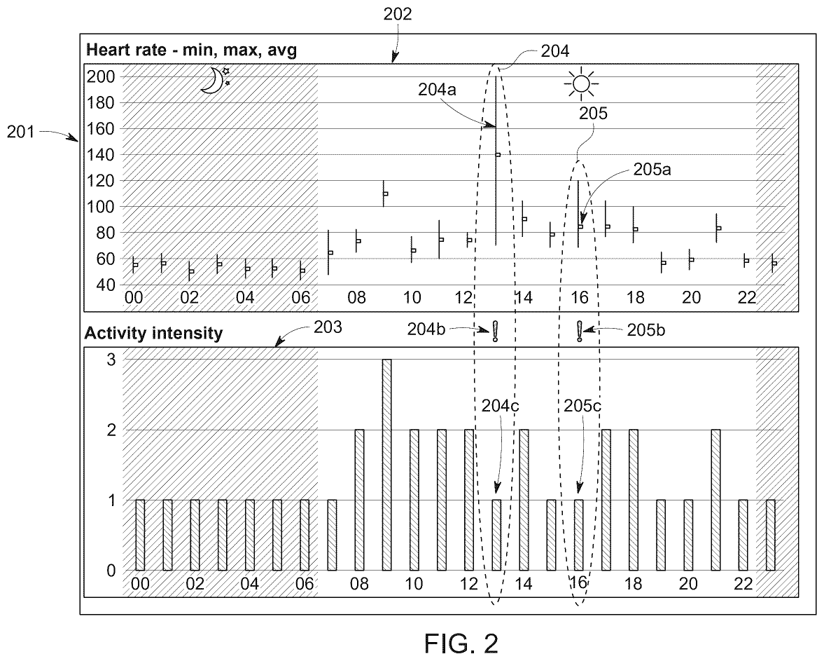

An example user interface 201 is shown in FIG. 2, for example displayed by display 107 to a clinician. As described herein, the example interface 201 of FIG. 2 may correspond to a “macro” display. The upper part or section 202 of interface 201 is an overview of a continuous patient metric, here the heart rate on a day. Within section 202 the lower part of each bar 204a displayed corresponds to the minimum heart rate in a particular hour, the upper part of the bar 204a is the maximum heart rate in that hour, and the dot 205a is the average heart rate in that hour.

The lower part or section 203 of user interface 201 shows activity data, here the activity intensity during the day. In section 203 each bar 204c, 205c displayed corresponds to one hour. The activity data is sorted into predetermined activity intensity levels or segments. In the example of FIG. 2, activity levels are 1 (resting), 2 (moderate intensity) or 3 (high intensity). Different scales or segmentation of intensity can be used.

In user interface 201 an exclamation mark 204b, which may be visually coded or otherwise distinguished from other data marks, indicates a clinical event, here an arrhythmia between 13:00 and 14:00. A different exclamation mark 205b indicates a different type of event, here discordant data, in this case between 16:00 and 17:00 the patient had a somewhat high heart rate while at rest, indicating discordance between the continuous patient metric and the activity data.

As illustrated, user interface 201 visually aligns certain elements for the user. In the example of FIG. 2, upper part 202 comprising continuous patient metric data is aligned in time with lower part 203 comprising activity data, filter into intensity categories. This visual alignment is highlighted for the purpose of illustration in FIG. 2 using dashed regions 204, 205, which may not be included in an embodiment.

In an embodiment, user interfaces such as user interface 201 allow for interaction. By way of example, user interfaces such as user interface 201 may be provided via an interactive website or smartphone app in which the user may interact, such as changing the time interval that is shown. In an embodiment, for example, by zooming out using an interface control element, system 100 can respond by filtering the data, such as querying database 106 or locally applying a data filter, to show multiple days with attendant visual updates, such as each bar or chart segment corresponding to one day. Similarly, by zooming in via user interaction, system 100 may filter the data to show the data per minute instead of hour, e.g., where each bar corresponds to one minute. Furthermore, when hovering over selected parts of user interface 201, for example an exclamation mark 204b, 205b, or a part of a heart rate bar 204a, system 100 may show the corresponding ECG strip. The user may also go to the previous or next period by e.g., by selecting a left arrow or right arrow or similar control element in user interface 201 (not shown).

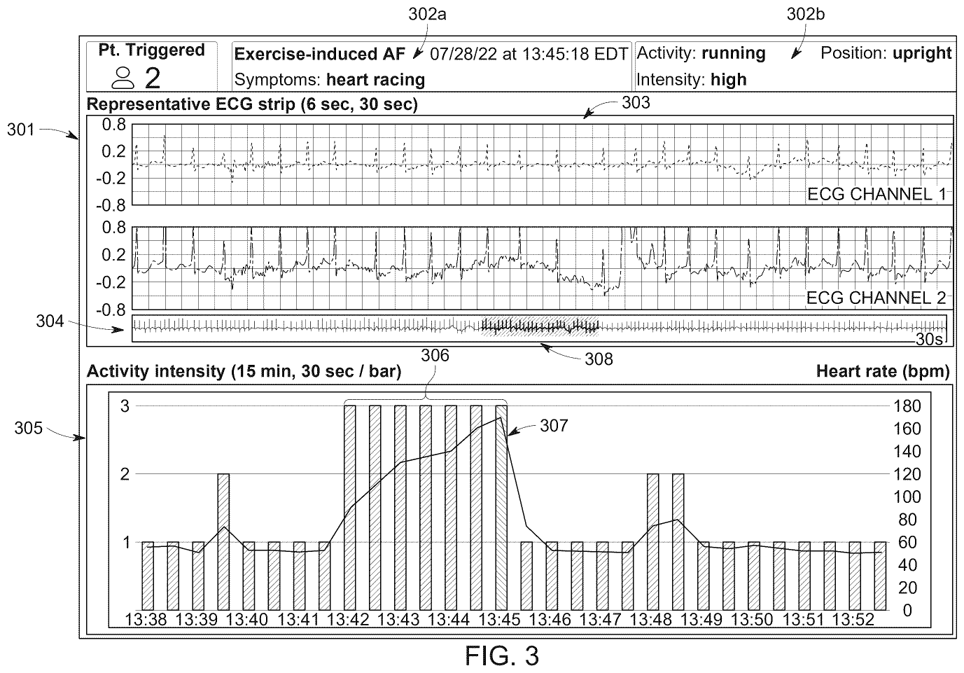

An example user interface 301 is shown in FIG. 3, for example displayed by display 107 to a clinician. As described herein, the example interface 301 of FIG. 3 may correspond to a “meso” display. A header part or section may include information in subparts 302a, 302b, such as patient data, time, sensor or system data, and insight data, such as activity category (e.g., running), position or orientation data (e.g., upright), intensity level (e.g., high), associated with the overall display or a subpart thereof, e.g., a highlighted or interacted with part of user interface 301. The upper part or section 303 of user interface 301 is a standard visualization of an ECG strip of 6 seconds in a 30-second fragment 304, with visual indication 308, such as a rectangle, indicating the position of the 6-second strip in fragment 304.

The lower part or section 305 of user interface 301 shows the activity intensity in a 15-minute period, where each bar, one of which is numbered 307, corresponds to the duration of fragment 304 (30 seconds). The highlighted bar 307 at 13:45 indicates the position of the shown ECG data in upper part 303 in the 15-minute period. Lower part or section 305 also shows the average heart rate per 30-second interval via a data mark in the form of a line graph.

In the example user interface 301, an arrhythmia took place at 13:45. Since clinicians also want to see how the arrhythmia starts and ends, a user may click on or interact with any bar, e.g., selected from among bars 306, in lower part or section 305, and as a result system 100 will show the corresponding 30-second fragment 304 in upper part or section 303. The user may also navigate from or within user interface 301 to differentially sort the displayed data, for example go to the previous or next ECG fragment or activity window by, e.g., selecting a control element such as a left arrow or right arrow (not shown). This permits the user to scroll the data horizontally through time.

It will be appreciated from review of FIG. 2 and FIG. 3 that system 100 may display user interface 301 in which first section 303 comprises electrocardiogram (ECG) strip (trace data) and ECG fragment 304 encompassing the ECG strip, with ECG strip and ECG fragment 304 being aligned in time with a subset, e.g., bar 307, of the activity data associated in time with the event. For example, a user may interact with an indicator of an event, such as indicator 204b of FIG. 2, to obtain user interface 301 as an updated contextual display, as illustrated in FIG. 3.

Within an updated contextual display, system 100 may respond, e.g., via the set of one or more processors of module 105a, to user interaction with the updated contextual display by visually indicating a second subset of the activity data and applying the one or more data filters to update the first section to display a second subset of the data for a continuous patient metric associated with the second subset of the activity data. By way of example, a user may interact with any of the bars, e.g., bars 306, in user interface 301 to obtain data filtered or sorted to visually associate different data of a continuous patient parameter, e.g., obtain from database 106 and displayed as ECG data in upper part or section 303 of user interface 301, corresponding to that associated in time with a selected bar in section 305.

System 100 may query and filter data to display in second section 305 comprising a plurality of data marks segmented according to time, e.g., as bars 306, 307, and displayed in areas of predetermined activity intensity, e.g., 1, 2, and 3, or some other predetermined intensity metric representative of a clinically significant patient activity or contextual data such as orientation or position. System 100, for example using the set of one or more processors of module 105a, is configured to respond to user interaction with one of the plurality of data marks by applying the one or more data filters to select and display updated data for upper section 303, e.g., ECG data that corresponds or is aligned in time with a selected bar in section 305.

System 100 may be configured to provide the updated contextual display using a second type of continuous patient metric, different than the initial type, displayed responsive to the interaction with an indicator of an event, e.g., indicators 204b, 205b. For example, the initial type of continuous patient metric may include one or more of heart rate data and blood pressure data, and the second type of continuous patient metric comprises electrocardiogram (ECG) data.

In an embodiment, the initial contextual display comprises an initial time filter applied to the activity data and the updated contextual display comprises the activity data filtered using a second time filter, different than the initial time filter, displayed responsive to the interaction with the indicator of the event or another interface tool, such as a zooming element. By way of example, an initial contextual display such as user interface 201 may be updated from displaying data activity in a day period, as in section 203, to displaying data activity in a 15-minute period, as in section 305.

In an embodiment, other user interactions may be used to apply predetermined data filtering for convenient display. By way of example, the set of one or more processors of module 105a may be configured to display, responsive to user interaction with the initial contextual display, such as in user interface 201, electrocardiogram (ECG) data associated with the event as an overlay on the initial contextual display. This permits a user to quickly retrieve relevant data of a continuous patient metric, such as ECG data, by interacting with an initially displayed activity data paired with a different continuous patient metric, such as heart rate. Similarly, system 100 may be configured to provide data filtering to display, responsive to user interaction with a part of the data for a continuous patient metric, such as heart rate in user interface 201, ECG data associated with the part. This permits a user to quickly retrieve relevant data of a continuous patient metric of a different type, such as ECG data, by interacting with an initially displayed continuous patent metric, such as heart rate.

It should be appreciated that various user controls are provided for filtering and sorting data in an interactive display in connection with a clinical event such as a cardiac event like an arrhythmia or another type of event, e.g., absence of arrhythmia and presence of data discordance. For example, in an embodiment, the event comprises one or more of a type of cardiac event and a discordance between the data for a continuous patient metric and the activity data. As illustrated in user interface 201, system 100 may be configured to display two or more indicators of events 204b, 205b in the initial contextual display.

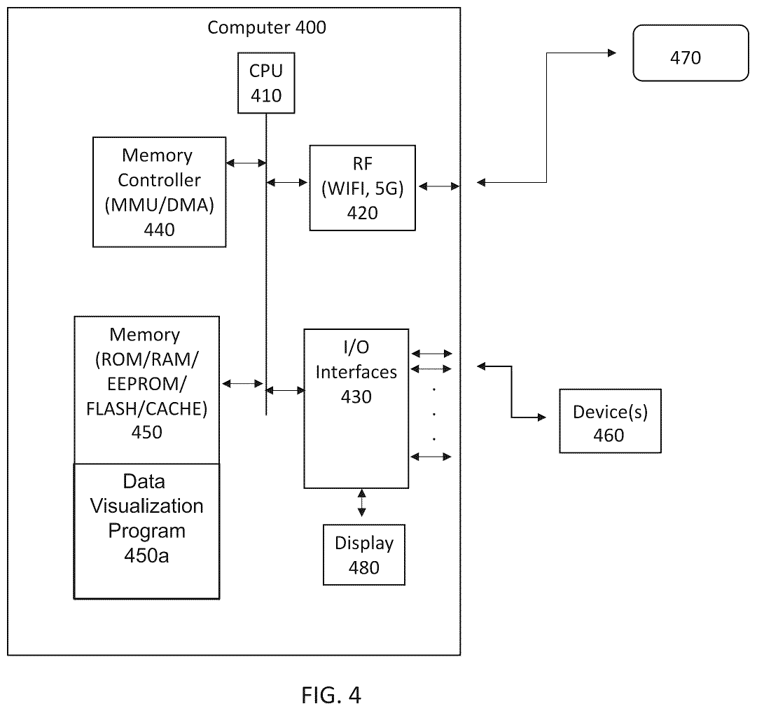

Referring to FIG. 4, it will be readily understood that certain embodiments can be implemented using any of a wide variety of devices or combinations of devices and components. In FIG. 4 an example of a computer 400 and its components are illustrated, which may be used in a device such as remote device 105 or local device 104 for implementing the functions or acts described herein, e.g., querying, filtering, or sorting of data for visualizations. Also, circuitry other than that illustrated in FIG. 4 may be utilized in one or more embodiments. The example of FIG. 4 includes certain functional blocks, as illustrated, which may be integrated onto a single semiconductor chip to meet specific application requirements.

One or more processing units are provided, which may include a central processing unit (CPU) 410, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 410 may comprise a single integrated circuit comprising several units, the design and arrangement of which vary according to the architecture chosen.

Computer 400 also includes a memory controller 440, e.g., comprising a direct memory access (DMA) controller to transfer data between memory 450 and hardware peripherals. Memory controller 440 includes a memory management unit (MMU) that functions to handle cache control, memory protection, and virtual memory. Computer 400 may include controllers for communication using various communication protocols (e.g., I²C, USB, etc.).

Memory 450 may include a variety of memory types, volatile and nonvolatile, e.g., read only memory (ROM), random access memory (RAM), electrically erasable programmable read only memory (EEPROM), Flash memory, and cache memory. Memory 450 may include embedded programs, code, and downloaded software, e.g., data visualization program 450a that provides coded methods such as illustrated and described in connection with FIG. 1A (or part thereof). By way of example, and not limitation, memory 450 may also include an operating system, application programs, other program modules, code, and program data, which may be downloaded, updated, or modified via remote devices.

A system bus permits communication between various components of the computer 400. I/O interfaces 430 and radio frequency (RF) devices 420, e.g., WIFI and telecommunication radios, may be included to permit computer 400 to send data to and receive data from remote devices using wireless mechanisms, noting that data exchange interfaces for wired data exchange may be utilized. Computer 400 may operate in a networked or distributed environment using logical connections to one or more other remote computers or devices 470, such as a database. The logical connections may include a network, such local area network (LAN) or a wide area network (WAN) but may also include other networks/buses. For example, computer 400 may communicate data with and between device(s) 460, for example personal user device(s) that provide communication and data connectivity to ambulatory patient sensors, etc.

Computer 400 may therefore execute program instructions or code configured to obtain, store, and analyze patient medical data and perform other functionality of the embodiments, such as described in connection with FIG. 1A. A user can interface with (for example, enter commands and information) the computer 400 through input devices, which may be connected to I/O interfaces 430. A display 480 or other type of output device may be connected to or integrated with the computer 400, for example via an interface selected from I/O interfaces 430.

It should be noted that the various functions described herein may be implemented using instructions or code stored on a memory, e.g., memory 450, that are transmitted to and executed by a processor, e.g., CPU 410. Computer 400 includes one or more storage devices that persistently store programs and other data. A storage device, as used herein, is a non-transitory computer readable storage medium. Some examples of a non-transitory storage device or computer readable storage medium include, but are not limited to, storage integral to computer 400, such as memory 450, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections using a mobile network, or through a hard wire connection, such as over a USB connection.

An embodiment may be implemented in a variety of devices, including user devices such as a smartphone or tablet running a mobile application. In one embodiment, referring to FIG. 1, the obtaining of patient data and employing a data visualization program 450a are performed locally on local device 104 such as a smartphone running a mobile application in module 104a. An embodiment may also be provided as a distributed system, where data is obtained by remote device 105 and functions and acts of data visualization program 450a are at least partially performed in remote device 105, e.g., by module 105a.

Therefore, an embodiment may include an application program configured to execute computer program instructions, for example as outlined at least in part in FIG. 1A, which in combination with local user device hardware, permit access to and visualization of medical data. Such an embodiment permits the data to be visualized on a variety of devices, e.g., display 107, remote device 105, or a device operatively coupled to one or more of the foregoing.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” or “the” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. The word “about” or similar relative term as applied to numbers includes ordinary (conventional) rounding of the number with a fixed base such as 5 or 10.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized or omitted as appropriate, as the explicit illustrated examples are used only for descriptive purposes and are not to be construed as limiting.

As used herein, the statement that two or more parts or components are “coupled” shall mean that the parts are joined or operate together either directly or indirectly, e.g., through one or more intermediate parts or components, so long as a link occurs. As used herein, “operatively coupled” means that two or more elements are coupled to operate together or are in communication, unidirectional or bidirectional, with one another. As used herein, the term “number” shall mean one or an integer greater than one (i.e., a plurality). As used herein a “set” shall mean one or more.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system, comprising:
an ambulatory patient sensor configured to be coupled to a patient and sense data for a continuous patient metric and activity data of the patient;
a local device in communication with the ambulatory patient sensor;
a remote device in communication with the local device;
a display in communication with the remote device; and
a database implemented in a non-transitory media;
the remote device comprising a set of one or more processors in communication with the database, the set of one or more processors configured to:
store the data for a continuous patient metric and the activity data of the patient in the database;
identify an event based on one or more of the data for a continuous patient metric and the activity data of the patient;
display, in a user interface provided at the display, an initial contextual display associated with the event and comprising an initial type of continuous patient metric and the activity data of the patient;
apply, based on interaction with the user interface, one or more data filters to the data for a continuous patient metric; and
thereafter provide an updated contextual display in the user interface comprising a first section having a subset of the data for a continuous patient metric aligned in time with a subset of the activity data in a second section.

2. The system of claim 1, wherein the first section comprises:
an electrocardiogram (ECG) strip; and
an ECG fragment encompassing the ECG strip;
the ECG strip and ECG fragment being aligned in time with the subset of the activity data associated with the event.

3. The system of claim 1, wherein the set of one or more processors is configured to respond to user interaction with the updated contextual display by:
visually indicating a second subset of the activity data; and
applying the one or more data filters to update the first section to display a second subset of the data for a continuous patient metric associated in time with the second subset of the activity data.

4. The system of claim 1, wherein the second section comprises a plurality of data marks segmented according to time and indicating amounts of predetermined activity intensity.

5. The system of claim 4, wherein the set of one or more processors is configured to respond to user interaction with one of the plurality of data marks by applying the one or more data filters to select and display updated data for the first section.

6. The system of claim 1, wherein the updated contextual display comprises a second type of continuous patient metric, different than the initial type, displayed responsive to the interaction with an indicator of the event.

7. The system of claim 6, wherein the initial type of continuous patient metric comprises one or more of heart rate data and blood pressure data, and wherein the second type of continuous patient metric comprises electrocardiogram (ECG) data.

8. The system of claim 1, wherein:
the initial contextual display comprises an initial time filter applied to the activity data; and
the updated contextual display comprises the activity data filtered using a second time filter, different than the initial time filter, displayed responsive to the interaction with the indicator of the event.

9. The system of claim 1, wherein:
the ambulatory patient sensor comprises a motion sensor; and
the activity data comprises data derived from the motion sensor.

10. The system of claim 1, wherein the set of one or more processors is configured to display, responsive to user interaction with the initial contextual display, electrocardiogram (ECG) data associated with the event as an overlay on the initial contextual display.

11. The system of claim 10, wherein the set of one or more processors is configured to display, responsive to user interaction with a part of the data for a continuous patient metric, electrocardiogram (ECG) data associated with the part.

12. The system of claim 1, wherein the event comprises one or more of a type of cardiac event and a discordance between the data for a continuous patient metric and the activity data.

13. The system of claim 12, wherein the set of one or more processors is configured to display two or more indicators of events in the initial contextual display.

14. A method, comprising:
obtaining, from an ambulatory patient sensor configured to be coupled to the patient, data for a continuous patient metric and activity data of the patient;
identifying, using a set of one or more processors, an event based on one or more of the data for a continuous patient metric and the activity data of the patient;
displaying, in a user interface, an initial contextual display associated with the event and comprising an initial type of continuous patient metric and the activity data of the patient;
applying, using the set of one or more processors, based on interaction with the user interface, one or more data filters to the data for a continuous patient metric; and
thereafter providing, using the set of one or more processors, an updated contextual display in the user interface comprising a first section having a subset of the data for a continuous patient metric aligned in time with a subset of the activity data in a second section.

15. A computer program product, comprising:
a non-transitory storage device comprising code executable by a set of one or more processors, the code comprising:
code that obtains, from an ambulatory patient sensor configured to be coupled to the patient, data for a continuous patient metric and activity data of the patient;
code that identifies an event based on one or more of the data for a continuous patient metric and the activity data of the patient;
code that provide data for display, in a user interface, of an initial contextual display associated with the event and comprising an initial type of continuous patient metric and the activity data of the patient;
code that applies, based on interaction with the user interface, one or more data filters to the data for a continuous patient metric; and
code that thereafter provides data for an updated contextual display in the user interface comprising a first section having a subset of the data for a continuous patient metric aligned in time with a subset of the activity data in a second section.
